(19) United States
(12) Patent Application Publication
(10) Pub. No.: US 2017/0291882 A1
(43) Pub. Date: Oct. 12, 2017

(54) Solid Forms of Mirabegron

(71) Applicant: Apotex Inc., Toronto (CA)

(72) Inventors: Allan W. Rey, Brantford (CA); Annyt Bhattacharyya, Hamilton (CA); Jenny L. Gerster, Brantford (CA); Sammy Chris Duncan, Brantford (CA); Mohammed Abdul Raheem, Brantford (CA); Fan Wang, Burlington (CA); Stuart P. Green, Mount Pleasant (CA)

(21) Appl. No.: 15/516,198

(22) PCT Filed: Oct. 1, 2015
(86) PCT No.: PCT/CA2015/000525

§ 371 Date: Mar. 31, 2017

Related U.S. Application Data
(60) Provisional application No. 62/058,296, filed on Oct. 1, 2014.

(51) Int. Cl. C07D 277/40; A61K 47/08; A61K 31/131; A61K 31/426; A61K 31/167

(52) U.S. Cl. CPC C07D 277/40; A61K 31/426; A61K 31/167; A61K 31/131; A61K 47/08; A61K 2300/00

(57) ABSTRACT

Solid forms of Mirabegron, including forms APO-I, APO-II, and various salt forms, and processes for the preparation thereof, are provided.

### TECHNICAL FIELD

The present invention is directed to Mirabegron and, in particular, to solid forms, including salt forms, thereof.

### BACKGROUND

Mirabegron (1) is a beta-3 adrenergic agonist. It is marketed in the United States as Myrbetriq™ and is indicated for the treatment of overactive bladder.

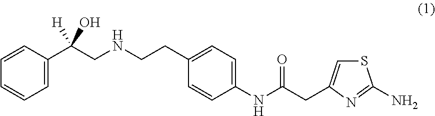 [SMILES: Nc1nc(CC(=O)Nc2ccc(CCNC[C@H](O)c3ccccc3)cc2)cs1]

U.S. Pat. No. 6,346,532 B1 discloses amide derivatives represented by general formula (I)

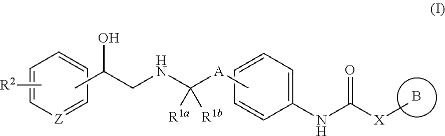

or salts thereof wherein each symbol has the following meaning: ring B: an optionally substituted heteroaryl optionally fused with a benzene ring; X: a bond, lower alkylene or lower alkenylene optionally substituted by hydroxy or lower alkyl, carbonyl, or a group represented by —NH— (when X is lower alkylene optionally substituted by lower alkyl which may be bonded to the hydrogen atom bonded to a constituent carbon atom of ring B to form lower alkylene to thereby form a ring); A: a lower alkylene or a group represented by -(lower alkylene)-O—; R^{1a} and R^{1b}: the same or different and each hydrogen or lower alkyl; R²: hydrogen or halogeno; and Z: nitrogen or a group represented by ═CH—. The compounds are useful as a diabetes remedy which not only functions to both accelerate the secretion of insulin and enhance insulin sensitivity but has an antiobestic action and an antihyperlipemic action based on its selective stimulative action on a β₃ receptor.

CA 2 464 068 C discloses novel crystals useful as a starting material for a diabetes remedy. These are α-form and β-form crystals of (R)-2-(2-aminothiazol-4-yl)-4′-[2-[(2-hydroxy-2-phenylethyl)amino]ethyl]acetanilide. The α-form crystal has no hygroscopicity, has stability which makes the crystal usable in a medicine, and is useful for mass synthesis in industrial production. The β-form crystal has relatively low hygroscopicity and is useful also as an intermediate for the β-form crystal.

JP 2011-105685 A discloses a crystal of (1R)-2-[(4-aminophenethyl) amino]-1-phenylethan-1-ol monohydrochloride (compound A) having suitable properties in industrial production. With respect to the crystal of compound A, the formed crystal unexpectedly varies in accordance with the difference in detailed production conditions and the compound A exhibits crystal polymorphism, and furthermore a crystal (type II crystal) different from the type I crystal of compound A is found. This type II crystal particularly excels in flowability and electrification and has suitable properties in industrial production.

WO 2012/156998 A2 relates to the amorphous form of Mirabegron, amorphous solid dispersion of Mirabegron, process for its preparation, processes for preparation of the a form crystal and the β form crystal of Mirabegron and a pharmaceutical composition thereof.

IP.com Journal 2013, 13(7A), 1-24 (IPCOM000228561D) discloses preparation of Mirabegron, its intermediates, a crystalline form of Mirabegron and a crystalline form of Mirabegron monohydrochloride.

IN 2012CH03843 A discloses a crystalline form of Mirabegron monohydrochloride and a crystalline form of Mirabegron, processes for their preparation and use thereof in a pharmaceutical composition.

IN 2012MU02588 A relates to Mirabegron and process of its preparation. In particular, it relates to an improved process for preparation of Mirabegron in amorphous or a crystalline form with better purity. The invention relates pharmaceutical compositions that include amorphous Mirabegron substantially free from crystalline forms. The invention also relates pharmaceutical compositions that include crystalline Mirabegron.

WO 2014/132270 A2 relates to a process for the preparation of 2-(2-aminothiazol-4-yl)-N-[4-(2-{[(2R)-2-hydroxy-2-phenylethyl]amino} ethyl)phenyl]acetamide monohydrochloride (Mirabegron hydrochloride), its intermediates and polymorph thereof.

IP.com Journal 2014, 15(1A), 1-6 (IPCOM000239988D) discloses crystalline forms of Mirabegron acetate designated as Form M1, Form M2, Form M3 and processes for their preparation thereof.

WO 2015/040605 A1 provides a crystalline form of Mirabegron, a process for its preparation, a pharmaceutical composition comprising it, and its use for the treatment of overactive bladder with symptoms of urinary incontinence, urgency, and urinary frequency.

EP 2 857 389 A1 relates to the acetate salt of Mirabegron, in particular in two novel crystalline forms, a process for their preparation and the use of said salt and its crystalline forms in the synthesis of Mirabegron with high yields and chemical purity.

WO 2015/044965 A1 relates to a process for the preparation of Mirabegron and alpha crystalline form thereof.

### SUMMARY

The present invention relates, at least in part, to form APO-I and APO-II of Mirabegron, salts of Mirabegron and processes for the preparation thereof. The forms of the present invention may have advantages relative to other known forms of Mirabegron, including, but not limited to, chemical stability, polymorphic stability, reduced hygroscopicity, varying solubility and/or bioavailability, varying morphology, and varying particle size. The Mirabegron solid forms of the present invention may have differential solubility with respect to undesired impurities which may allow for improved purification from impurities when compared to other solid forms of Mirabegron.

In illustrative embodiments, there is provided form APO-I of Mirabegron characterized by a PXRD diffractogram comprising a peak, in terms of degrees 2θ, at 7.4+/−0.2, in addition to at least three peaks, in terms of degrees 2θ, selected from the group consisting of: 16.3+/−0.2, 16.8+/−0.2, 19.8+/−0.2, 21.2+/−0.2, 24.3+/−0.2, and 25.6+/−0.2.

In illustrative embodiments, there is provided a form APO-I of Mirabegron described herein wherein the PXRD diffractogram further comprises a peak, in terms of degrees 2θ, at 8.9+/−0.2.

In illustrative embodiments, there is provided a form APO-I of Mirabegron described herein wherein the PXRD diffractogram further comprises peaks, in terms of degrees 2θ, at 7.4+/−0.2, 8.9+/−0.2, 16.3+/−0.2, 16.8+/−0.2, and 24.3+/−0.2.

In illustrative embodiments, there is provided a form APO-I of Mirabegron described herein characterized by a PXRD diffractogram substantially similar to the diffractogram as depicted in FIG. 1.

In illustrative embodiments, there is provided a form APO-I of Mirabegron described herein wherein the form APO-I is a solvate with (S)-(+)-1,2-propanediol having a molar ratio of Mirabegron to (S)-(+)-1,2-propanediol of 1:1.

In illustrative embodiments, there is provided form APO-II of Mirabegron characterized by a PXRD diffractogram comprising peaks, in terms of degrees 2θ, at 9.7+/−0.2, 15.5+/−0.2, 17.1+/−0.2, and 18.5+/−0.2 and at least one peak selected from the group consisting of: 12.3+/−0.2 and 13.8+/−0.2.

In illustrative embodiments, there is provided a form APO-II of Mirabegron described herein characterized by a PXRD diffractogram substantially similar to the diffractogram as depicted in FIG. 2.

In illustrative embodiments, there is provided a form APO-II of Mirabegron described herein wherein the form APO-II is a solvate with (R)-(−)-1,2-propanediol having a molar ratio of Mirabegron to (R)-(−)-1,2-propanediol of 1:1.

In illustrative embodiments, there is provided a process for the preparation of form APO-I of Mirabegron comprising treating a mixture of Mirabegron in a solvent comprising (S)-(+)-1,2-propanediol with an antisolvent to provide form APO-I of Mirabegron.

In illustrative embodiments, there is provided a process described herein wherein the mixture of Mirabegron is in the form of a solution of Mirabegron.

In illustrative embodiments, there is provided a process described herein wherein the mixture of Mirabegron is in the form of a suspension of Mirabegron in a solvent comprising (S)-(+)-1,2-propanediol that is seeded with form APO-I of Mirabegron.

In illustrative embodiments, there is provided a process described herein wherein the solvent is a mixture of (S)-(+)-1,2-propanediol and one or more solvents selected from ketones, esters, cyclic ethers, and n-butanol.

In illustrative embodiments, there is provided a process described herein wherein the solvent is a mixture of (S)-(+)-1,2-propanediol and one or more solvents selected from methyl isobutyl ketone, ethyl acetate, isopropyl acetate, 2-methyltetrahydrofuran and n-butanol.

In illustrative embodiments, there is provided a process described herein wherein the solvent is (S)-(+)-1,2-propanediol.

In illustrative embodiments, there is provided a process described herein wherein the antisolvent is methyl t-butyl ether.

In illustrative embodiments, there is provided a process described herein wherein the solvent is a mixture of (S)-(+)-1,2-propanediol and 2-methyltetrahydrofuran, the antisolvent is methyl t-butyl ether, and the treating is done at a temperature of between about 20° C. and about 30° C.

In illustrative embodiments, there is provided a process for the preparation of form APO-I of Mirabegron comprising:
a) obtaining a solution of Mirabegron in (S)-(+)-1,2-propanediol at about 65° C.;
b) adding from about 35 volumes to about 50 volumes of methyl t-butyl ether to the solution while cooling the solution from about 65° C. to less than about 30° C. at a rate of at least about 25° C./hour, thereby forming a suspension; and
c) filtering the suspension thereby isolating from APO-I of Mirabegron.

In illustrative embodiments, there is provided a process described herein wherein the solution is seeded with form APO-I.

In illustrative embodiments, there is provided a process for the preparation of form APO-II of Mirabegron comprising:
a) obtaining a solution of Mirabegron in (R)-(−)-1,2-propanediol at about 65° C.;
b) adding from about 35 volumes to about 50 volumes of methyl t-butyl ether to the solution while cooling the solution from about 65° C. to less than about 30° C. at a rate of at least about 25° C./hour, thereby forming a suspension; and
c) filtering the suspension thereby isolating form APO-II of Mirabegron.

In illustrative embodiments, there is provided a process described herein wherein the solution is seeded with form APO-II.

In illustrative embodiments, there is provided a hydrobromide salt of Mirabegron.

In illustrative embodiments, there is provided a hydrobromide salt of Mirabegron as described herein characterized by a PXRD diffractogram comprising peaks, in terms of degrees 2θ, at 6.5+/−0.2, 8.7+/−0.2, 20.2+/−0.2, and 24.1+/−0.2.

In illustrative embodiments, there is provided a monotartrate salt of Mirabegron.

In illustrative embodiments, there is provided a monotartrate salt of Mirabegron as described herein characterized by a PXRD diffractogram comprising peaks, in terms of degrees 2θ, at 5.2+/−0.2, 8.0+/−0.2, 15.2+/−0.2, and 19.5+/−0.2.

In illustrative embodiments, there is provided a benzoate salt of Mirabegron.

In illustrative embodiments, there is provided a benzoate salt of Mirabegron as described herein characterized by a PXRD diffractogram comprising peaks, in terms of degrees 2θ, at 5.9+/−0.2, 9.1+/−0.2, 16.5+/−0.2, and 18.2+/−0.2.

In illustrative embodiments, there is provided an oxalate salt of Mirabegron.

In illustrative embodiments, there is provided an oxalate salt of Mirabegron as described herein characterized by a PXRD diffractogram comprising peaks, in terms of degrees 2θ, at 6.6+/−0.2, 9.2+/−0.2, 15.5+/−0.2, and 19.7+/−0.2.

In illustrative embodiments, there is provided a monosuccinate salt of Mirabegron.

In illustrative embodiments, there is provided a monosuccinate salt of Mirabegron as described herein characterized by a PXRD diffractogram comprising peaks, in terms of degrees 2θ, at 5.3+/−0.2, 8.1+/−0.2, 15.3+/−0.2, and 19.1+/−0.2.

In illustrative embodiments, there is provided a p-toluenesulfonate salt of Mirabegron.

In illustrative embodiments, there is provided a p-toluenesulfonate salt of Mirabegron as described herein characterized by a PXRD diffractogram comprising peaks, in terms of degrees 2θ, at 8.1+/−0.2, 9.9+/−0.2, 17.2+/−0.2, and 18.0+/−0.2.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

In drawings which illustrate embodiments of the invention,

FIG. 1 is a PXRD diffractogram of form APO-I of Mirabegron as prepared in Example 1.

FIG. 2 is a PXRD diffractogram of form APO-II of Mirabegron as prepared in Example 4.

FIG. 3 is a PXRD diffractogram of form APO-II of Mirabegron as prepared in Example 5.

FIG. 4 is a PXRD diffractogram of the hydrobromide salt of Mirabegron as prepared in Example 8.

FIG. 5 is a PXRD diffractogram of the monotartrate salt of Mirabegron as prepared in Example 9.

FIG. 6 is a PXRD diffractogram of the benzoate salt of Mirabegron as prepared in Example 10.

FIG. 7 is a PXRD diffractogram of the oxalate salt of Mirabegron as prepared in Example 11.

FIG. 8 is a PXRD diffractogram of the monosuccinate salt of Mirabegron as prepared in Example 12.

FIG. 9 is a PXRD diffractogram of the p-toluenesulfonate salt of Mirabegron as prepared in Example 13.

### DETAILED DESCRIPTION

When used in reference to a diffractogram, a spectrum and/or data presented in a graph, the term “substantially similar” means that the subject diffractogram, spectrum and/or data presented in a graph encompasses all diffractograms, spectra and/or data presented in graphs that vary within acceptable boundaries of experimentation that are known to a person of skill in the art. Such boundaries of experimentation will vary depending on the type of the subject diffractogram, spectrum and/or data presented in a graph, but will nevertheless be known to a person of skill in the art.

When used in reference to a peak in a PXRD diffractogram, the term “approximately” means that the peak may vary by ±0.2 degrees 2θ of the subject value.

As used herein, the term “about” means close to and that variation from the exact value that follows the term within amounts that a person of skill in the art would understand to be reasonable. In particular, when the term “about” is used with respect to temperature, a variation of +/−5° C. is often acceptable.

As used herein, the term “volumes” refers to the parts of solvent or liquids by volume (mL) with respect to the weight of solute (g). For example, when an experiment is conducted using 1 g of starting material and 100 mL of solvent, it is said that 100 volumes of solvent are used.

As used herein, when referring to a diffractogram, spectrum and/or to data presented in a graph, the term “peak” refers to a feature that one skilled in the art would recognize as not attributable to background noise.

Depending on the nature of the methodology applied and the scale selected to display results obtained from an X-ray diffraction analysis, an intensity of a peak obtained may vary quite dramatically. For example, it is possible to obtain a relative peak intensity of 1% when analyzing one sample of a substance, but another sample of the same substance may show a much different relative intensity for a peak at the same position. This may be due, in part, to the preferred orientation of the sample and its deviation from the ideal random sample orientation, sample preparation and the methodology applied. Such variations are known and understood by a person of skill in the art.

Multi-component solid forms comprising more than one type of molecule, such as solvates may have some variability in the exact molar ratio of their components depending on a variety of conditions understood to a person of skill in the art. For example, a molar ratio of components within a solvate provides a person of skill in the art information as to the general relative quantities of the components of the solvate and, in many cases, the molar ratio may vary by plus or minus 20% from a stated range. For example, a molar ratio of 1:1 is understood to include the ratio 1:0.8 as well as 1:1.2 as well as all of the individual ratios in between.

In an embodiment, the present invention provides form APO-I of Mirabegron which may be characterized by a PXRD diffractogram comprising a peak, in terms of degrees 2θ, at 7.4+/−0.2, in addition to at least three peaks, in terms of degrees 2θ, selected from the group consisting of: 16.3+/−0.2, 16.8+/−0.2, 19.8+/−0.2, 21.2+/−0.2, 24.3+/−0.2, and 25.6+/−0.2.

In an embodiment, the present invention provides form APO-I of Mirabegron which may be characterized by a PXRD diffractogram comprising peaks, in terms of degrees 2θ, at 7.4+/−0.2 and 8.9+/−0.2, in addition to at least three peaks, in terms of degrees 2θ, selected from the group consisting of: 16.3+/−0.2, 16.8+/−0.2, 19.8+/−0.2, 21.2+/−0.2, 24.3+/−0.2, and 25.6+/−0.2.

In an embodiment, the present invention provides form APO-I of Mirabegron characterized by a PXRD diffractogram comprising peaks, in terms of degrees 2θ, at 7.4+/−0.2, 8.9+/−0.2, 16.3+/−0.2, 16.8+/−0.2, and 24.3+/−0.2.

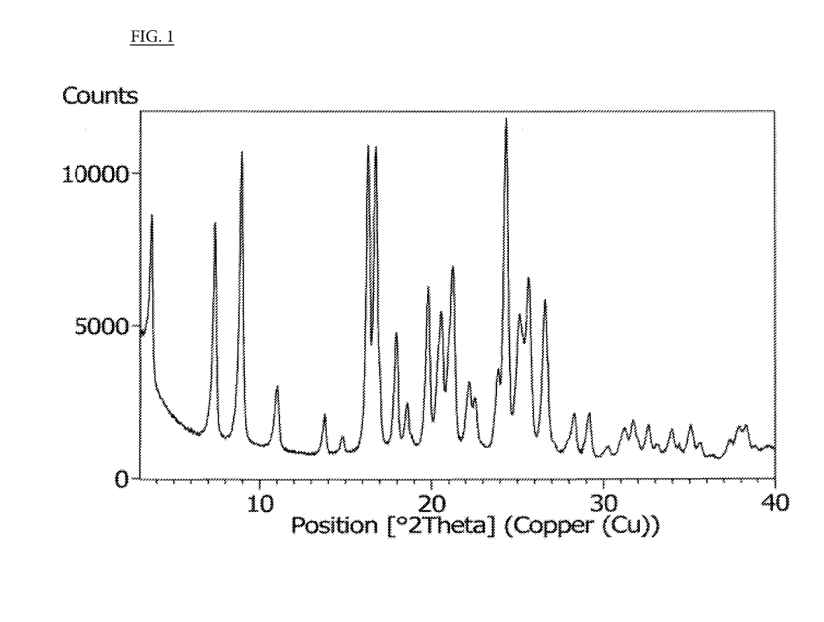

An illustrative PXRD diffractogram of form APO-I Mirabegron is shown in FIG. 1. Form APO-1 may have a peak at any one or more of the values expressed in degrees 2θ given in Table 1. Although values are given in the table below, APO-I may be defined by the claimed peaks and a particular claim may be limited to one peak only, or several peaks. The form APO-I does not have to include all or even many of the peaks listed in Table 1. Some illustrative and non-limiting possible observations regarding relative intensities of the peaks are set out in Table 1.

TABLE 1
Relative peak intensities of form APO-I

| Angle (° 2θ) | Relative intensity (%) |
|---|---|
| 3.65 | 46.07 |
| 7.36 | 68.06 |
| 8.91 | 88.73 |
| 11.05 | 6.16 |
| 13.77 | 12.16 |
| 14.82 | 4.78 |
| 16.32 | 90.97 |
| 16.76 | 87.85 |
| 17.94 | 34.25 |
| 18.58 | 13.95 |
| 19.79 | 49.79 |
| 20.41 | 14.19 |
| 20.59 | 32.53 |
| 21.19 | 54.99 |
| 22.08 | 13.73 |
| 22.21 | 11.16 |
| 22.53 | 18.50 |
| 23.85 | 30.67 |
| 24.33 | 100.00 |
| 25.14 | 38.93 |
| 25.64 | 48.85 |
| 26.65 | 34.80 |
| 28.23 | 12.47 |

In an embodiment, form APO-I of the present invention is a solvate with (S)-(+)-1,2-propanediol wherein the molar ratio of Mirabegron to (S)-(+)-1,2-propanediol is 1:1.

In an embodiment, the present invention provides form APO-II of Mirabegron which may be characterized by a PXRD diffractogram comprising at least one peak, in terms of degrees 2θ, selected from the group consisting of: 12.3+/−0.2 and 13.8+/−0.2, in addition to peaks, in terms of degrees 2θ, at 9.7+/−0.2, 15.5+/−0.2, 17.1+/−0.2, and 18.5+/−0.2.

In an embodiment, the present invention provides form APO-II of Mirabegron which may be characterized by a PXRD diffractogram comprising peaks, in terms of degrees 2θ, at 9.7+/−0.2, 12.3+/−0.2, 15.5+/−0.2, 17.1+/−0.2, and 18.5+/−0.2.

In an embodiment, the present invention provides form APO-II of Mirabegron which may be characterized by a PXRD diffractogram comprising peaks, in terms of degrees 2θ, at 9.7+/−0.2, 13.8+/−0.2, 15.5+/−0.2, 17.1+/−0.2, and 18.5+/−0.2.

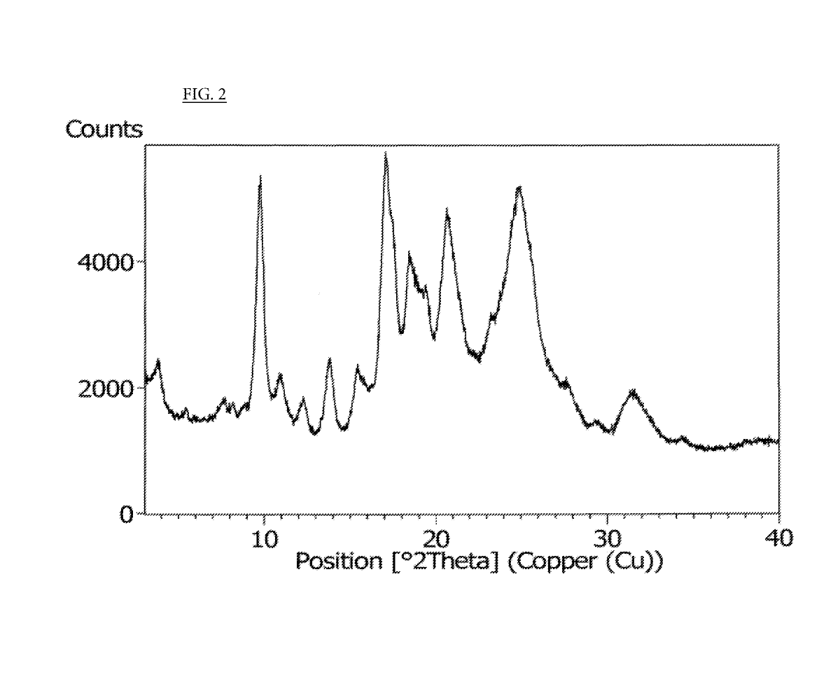

An illustrative PXRD diffractogram of form APO-II is shown in FIG. 2. Form APO-II may have a peak at any one or more of the values expressed in degrees 2θ given in Table 2. Although values are given in the table below, APO-II may be defined by the claimed peaks and a particular claim may be limited to one peak only, or several peaks. The form APO-II does not have to include all or even many of the peaks listed in Table 2. Some illustrative and non-limiting possible observations regarding relative intensities of the peaks are set out in Table 2.

TABLE 2
Relative peak intensities of form APO-II

| Angle (° 2θ) | Relative intensity (%) |
|---|---|
| 9.74 | 100.00 |
| 12.25 | 15.36 |
| 13.79 | 33.99 |
| 15.47 | 21.34 |
| 17.07 | 85.70 |
| 18.49 | 56.77 |
| 24.89 | 72.28 |
| 28.00 | 15.33 |

In an embodiment, form APO-II of the present invention is a solvate with (R)-(−)-1,2-propanediol wherein the molar ratio of Mirabegron to (R)-(−)-1,2-propanediol is 1:1.

A process is provided for the preparation of form APO-I of Mirabegron comprising:
a) obtaining a solution of Mirabegron in (S)-(+)-1,2-propanediol at about 65° C.;
b) adding from about 35 volumes to about 50 volumes of methyl t-butyl ether to the solution while cooling the solution from about 65° C. to less than about 30° C. at a rate of at least about 25° C./hour thereby forming a suspension; and
c) filtering the suspension thereby isolating form APO-I of Mirabegron.

Often the process comprises obtaining a solution at 65° C. Often the volume of methyl t-butyl ether added is from 35 volumes to 50 volumes. Often the rate of cooling is 25° C./hour.

Another process is provided for the preparation of form APO-I of Mirabegron comprising treating a mixture of Mirabegron in a solvent comprising (S)-(+)-1,2-propanediol with an antisolvent to provide form APO-I of Mirabegron. The solution in this process may be formed from isolated Mirabegron or it may be obtained from a previous reaction step wherein Mirabegron is generated, but not isolated. The solvent in this process comprises (S)-(+)-1,2-propanediol, optionally as part of a mixture with one or more solvents selected from the group consisting of n-butanol, esters such as ethyl acetate and isopropyl acetate, ketones such as acetone and methyl iso-butyl ketone, and cyclic ethers such as 2-methyl tetrahydrofuran. In preferred embodiments of the process, the solvent is comprised solely of (S)-(+)-1,2-propanediol, or is a mixture of (S)-(+)-1,2-propanediol and 2-methyltetrahydrofuran. Within the process, the amount of (S)-(+)-1,2-propanediol is at least about 1 mole equivalent with respect to the amount of Mirabegron. When the solvent is a mixture of (S)-(+)-1,2-propanediol and other solvents, the amount of the other solvent used may be varied, but is generally from about 1 volume to about 10 volumes relative to the amount of (S)-(+)-1,2-propanediol.

When the Mirabegron solution is formed from isolated Mirabegron, a heating step (to a temperature of about 70° C. to about 80° C.) may be required to obtain complete dissolution. When the solution is obtained from a previous reaction step, the heating step may not be required.

Seeding the Mirabegron solution with at least about 1 wt % of form APO-I Mirabegron can be employed prior to addition of antisolvent. A preferred antisolvent is methyl t-butyl ether. The amount of antisolvent may vary depending on the corresponding amount of solvent, but is generally from about 10 volumes to about 60 volumes relative to the amount of solvent, and may be added portion wise or in a single portion. The antisolvent may be added before, during or after cooling a hot solution of Mirabegron to the isolation temperature, preferably at a temperature of between 20° C. to about 30° C. The isolated form APO-I Mirabegron may be washed with an antisolvent to remove excess residual (S)-(+)-1,2-propanediol and dried at a temperature below about 60° C.

Alternatively, the Mirabegron mixture may be a suspension of Mirabegron obtained from seeding a solution of Mirabegron in a solvent comprising (S)-(+)-1,2-propanediol with at least about 1 wt % of form APO-I seeds. Often, the suspension is a creamy suspension obtained while maintaining a seeded solution of Mirabegron for at least about 30 minutes. The solution may be formed from isolated Mirabegron or it may be obtained from a previous reaction step wherein Mirabegron is generated, but not isolated. The solvent used for the solution comprises (S)-(+)-1,2-propanediol, optionally as part of a mixture with one or more solvents selected from the group consisting of n-butanol, esters such as ethyl acetate and isopropyl acetate, ketones such as acetone and methyl iso-butyl ketone, and cyclic ethers such as 2-methyl tetrahydrofuran.

In preferred embodiments of the process, the solvent is comprised solely of (S)-(+)-1,2-propanediol, or is a mixture of (S)-(+)-1,2-propanediol and 2-methyltetrahydrofuran. Within the process, the amount of (S)-(+)-1,2-propanediol is at least about 1 mole equivalent with respect to the amount of Mirabegron. When the solvent is a mixture of (S)-(+)-1,2-propanediol and other solvents, the amount of the other solvent used may be varied, but is generally from about 1 volume to about 10 volumes relative to the amount of (S)-(+)-1,2-propanediol. When the Mirabegron solution is formed from isolated Mirabegron, a heating step (to a temperature of about 70° C. to about 80° C.) may be required to obtain complete dissolution. When the solution is obtained from a previous reaction step, the heating step may not be required.

A preferred antisolvent for use in the process is methyl t-butyl ether. The amount of antisolvent may vary depending on the corresponding amount of solvent, but is generally from about 10 volumes to about 60 volumes relative to the amount of solvent, and may be added portion wise or in a single portion. The antisolvent may be added to the suspension of Mirabegron at a temperature from about 20° C. to about 80° C., preferably at a temperature of between about 20° C. to about 30° C. The isolated form APO-I Mirabegron may be washed with an antisolvent to remove excess residual (S)-(+)-1,2-propanediol and dried at a temperature below about 60° C.

A process is provided for the preparation of form APO-II of Mirabegron comprising:
a) obtaining a solution of Mirabegron in (R)-(−)-1,2-propanediol at about 65° C.;
b) adding from about 35 volumes to about 50 volumes of methyl t-butyl ether to the solution while cooling the solution from about 65° C. to less than about 30° C. at a rate of at least about 25° C./hour thereby forming a suspension; and
c) filtering the suspension thereby isolating form APO-II of Mirabegron.

Often the process is comprises obtaining a solution at 65° C. Often the volume of methyl t-butyl ether added is from 35 volumes to 50 volumes. Often the rate of cooling is 25° C./hour.

In an embodiment, the present invention provides a hydrobromide salt of Mirabegron.

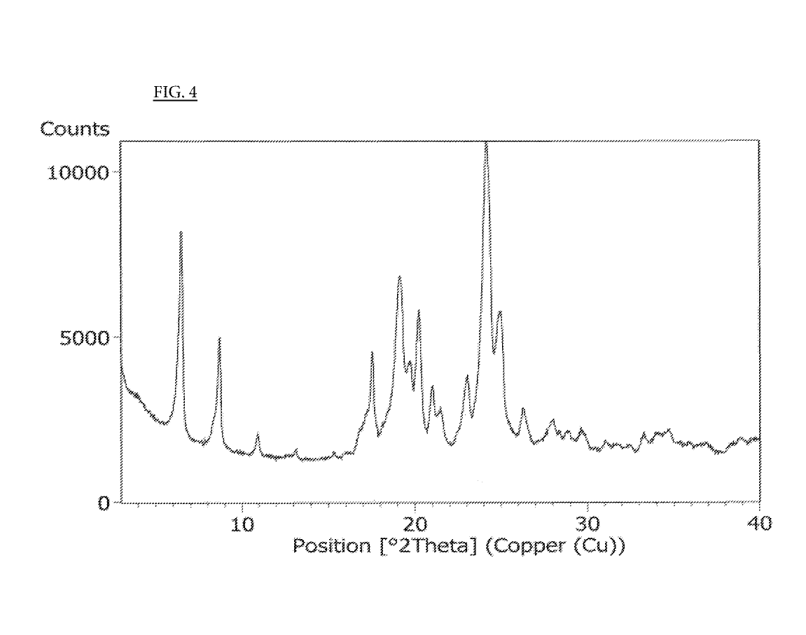

In an embodiment, the present invention provides a hydrobromide salt characterized by a PXRD diffractogram comprising peaks, in terms of degrees 2θ, at 6.5+/−0.2, 8.7+/−0.2, 20.2+/−0.2, and 24.1+/−0.2. An illustrative PXRD diffractogram of the hydrobromide salt of Mirabegron is shown in FIG. 4.

The hydrobromide salt of Mirabegron may have a peak at any one or more of the values expressed in degrees 2θ given in Table 3. Although values are given in the table below, the hydrobromide salt may be defined by the claimed peaks and a particular claim may be limited to one peak only, or several peaks. The hydrobromide salt does not have to include all or even many of the peaks listed in Table 3. Some illustrative and non-limiting possible observations regarding relative intensities of the peaks are set out in Table 3.

TABLE 3
Relative peak intensities of the
hydrobromide salt of Mirabegron

| Angle (° 2θ) | Relative intensity (%) |
|---|---|
| 6.45 | 85.37 |
| 8.65 | 51.13 |
| 10.88 | 9.91 |
| 17.53 | 40.10 |
| 19.04 | 37.56 |
| 19.68 | 25.79 |
| 20.21 | 48.87 |
| 20.99 | 19.97 |
| 22.99 | 20.68 |
| 24.08 | 100.00 |
| 24.84 | 25.48 |
| 26.28 | 14.38 |

In an embodiment, the present invention provides a monotartrate salt of Mirabegron.

In an embodiment, the present invention provides a monotartrate salt characterized by a PXRD diffractogram comprising peaks, in terms of degrees 2θ, at 5.2+/−0.2, 8.0+/−0.2, 15.2+/−0.2, and 19.5+/−0.2.

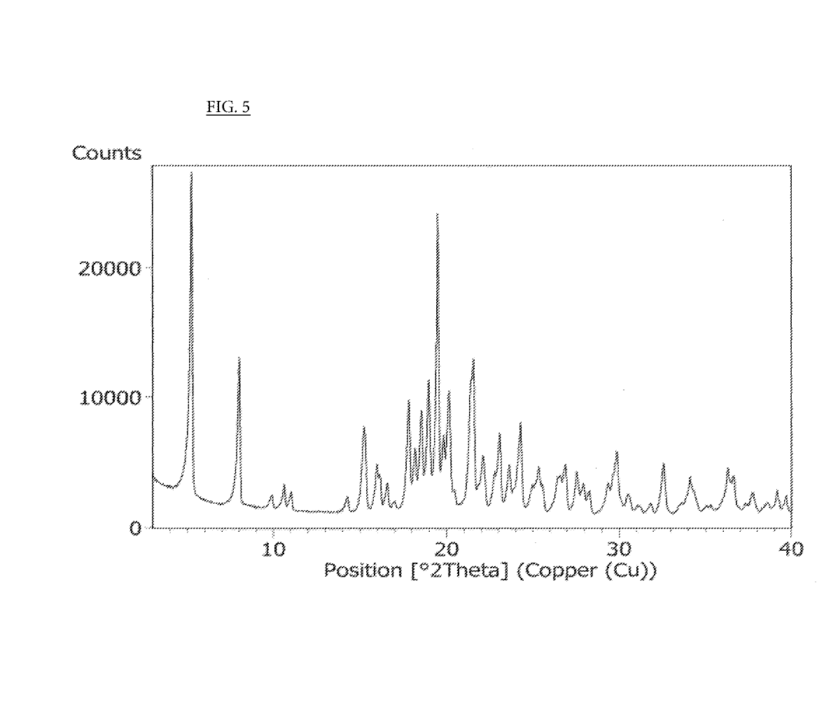

An illustrative PXRD diffractogram of the monotartrate salt of Mirabegron is shown in FIG. 5.

The monotartrate salt of Mirabegron may have a peak at any one or more of the values expressed in degrees 2θ given in Table 4. Although values are given in the table below, the monotartrate salt may be defined by the claimed peaks and a particular claim may be limited to one peak only, or several peaks. The monotartrate salt does not have to include all or even many of the peaks listed in Table 4. Some illustrative and non-limiting possible observations regarding relative intensities of the peaks are set out in Table 4.

TABLE 4
Relative peak intensities of the
monotartrate salt of Mirabegron

| Angle (° 2θ) | Relative intensity (%) |
|---|---|
| 5.22 | 100.00 |
| 7.97 | 46.25 |
| 9.84 | 4.58 |
| 10.57 | 7.89 |
| 10.98 | 5.54 |
| 14.22 | 4.65 |
| 15.21 | 26.22 |
| 15.98 | 13.37 |
| 16.54 | 8.89 |
| 17.79 | 31.58 |
| 18.16 | 13.58 |
| 18.52 | 27.15 |
| 18.94 | 37.81 |
| 19.47 | 96.25 |
| 19.82 | 18.50 |
| 20.11 | 35.54 |
| 21.41 | 38.19 |
| 22.08 | 14.91 |
| 23.05 | 24.59 |
| 23.62 | 13.09 |
| 24.21 | 25.86 |

In an embodiment, the present invention provides a benzoate salt of Mirabegron.

In an embodiment, the present invention provides a benzoate salt characterized by a PXRD diffractogram comprising peaks, in terms of degrees 2θ, at 5.9+/−0.2, 9.1+/−0.2, 16.5+/−0.2, and 18.2+/−0.2.

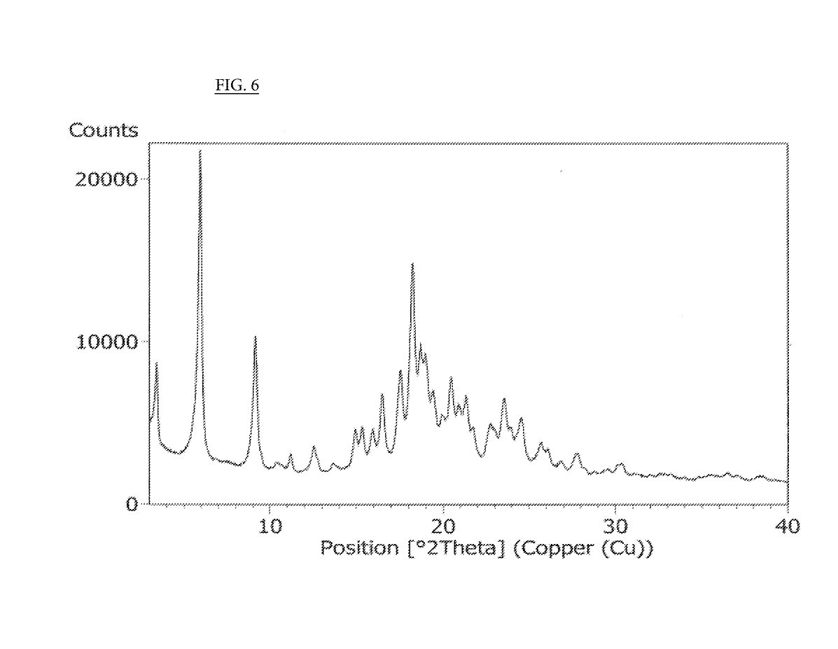

An illustrative PXRD diffractogram of the benzoate salt of Mirabegron is shown in FIG. 6.

The benzoate salt of Mirabegron may have a peak at any one or more of the values expressed in degrees 26 given in Table 5. Although values are given in the table below, the benzoate salt may be defined by the claimed peaks and a particular claim may be limited to one peak only, or several peaks. The benzoate salt does not have to include all or even many of the peaks listed in Table 5. Some illustrative and non-limiting possible observations regarding relative intensities of the peaks are set out in Table 5.

TABLE 5
Relative peak intensities of the
benzoate salt of Mirabegron

| Angle (°2θ) | Relative intensity (%) |
|---|---|
| 5.93 | 100.00 |
| 9.12 | 42.68 |
| 12.52 | 8.31 |
| 14.91 | 11.40 |
| 15.31 | 10.69 |
| 15.90 | 10.10 |
| 16.47 | 20.76 |
| 17.50 | 27.32 |
| 18.22 | 59.50 |
| 18.68 | 24.44 |
| 20.46 | 20.61 |
| 21.31 | 16.29 |
| 23.51 | 17.78 |

In an embodiment, the present invention provides an oxalate salt of Mirabegron.

In an embodiment, the present invention provides an oxalate salt characterized by a PXRD diffractogram comprising peaks, in terms of degrees 2θ, at 6.6+/−0.2, 9.2+/−0.2, 15.5+/−0.2, and 19.7+/−0.2.

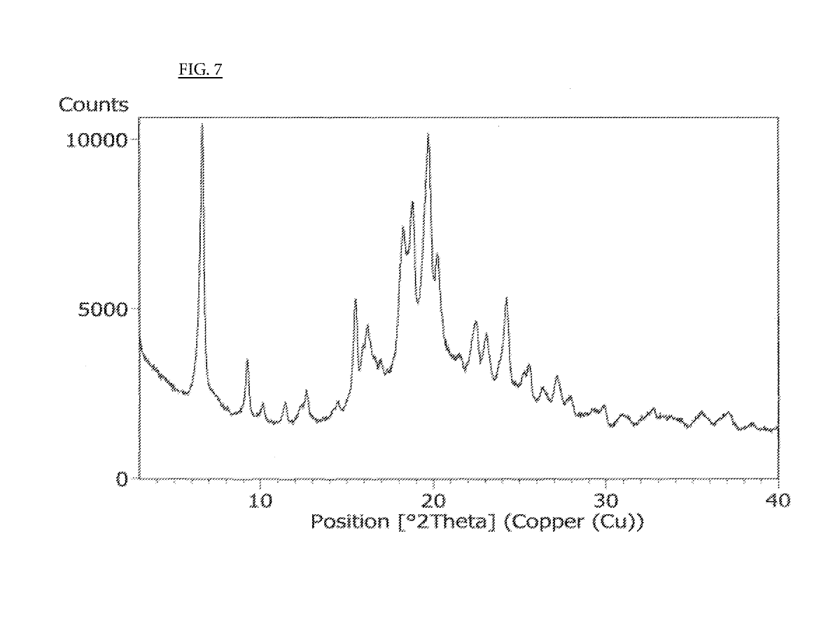

An illustrative PXRD diffractogram of the oxalate salt of Mirabegron is shown in FIG. 7.

The oxalate salt of Mirabegron may have a peak at any one or more of the values expressed in degrees 2θ given in Table 6. Although values are given in the table below, the oxalate salt may be defined by the claimed peaks and a particular claim may be limited to one peak only, or several peaks. The oxalate salt does not have to include all or even many of the peaks listed in Table 6. Some illustrative and non-limiting possible observations regarding relative intensities of the peaks are set out in Table 6.

TABLE 6
Relative peak intensities of the
oxalate salt of Mirabegron

| Angle (° 2θ) | Relative intensity (%) |
|---|---|
| 6.62 | 100.00 |
| 9.23 | 22.04 |
| 11.40 | 9.01 |
| 15.47 | 35.91 |
| 18.30 | 8.74 |
| 18.79 | 37.09 |
| 19.69 | 77.26 |
| 20.26 | 24.77 |
| 22.40 | 25.26 |
| 23.06 | 14.75 |
| 24.21 | 38.92 |

In an embodiment, the present invention provides a monosuccinate salt of Mirabegron.

In an embodiment, the present invention provides a monosuccinate salt characterized by a PXRD diffractogram comprising peaks, in terms of degrees 2θ, at 5.3+/−0.2, 8.1+/−0.2, 15.3+/−0.2, and 19.1+/−0.2.

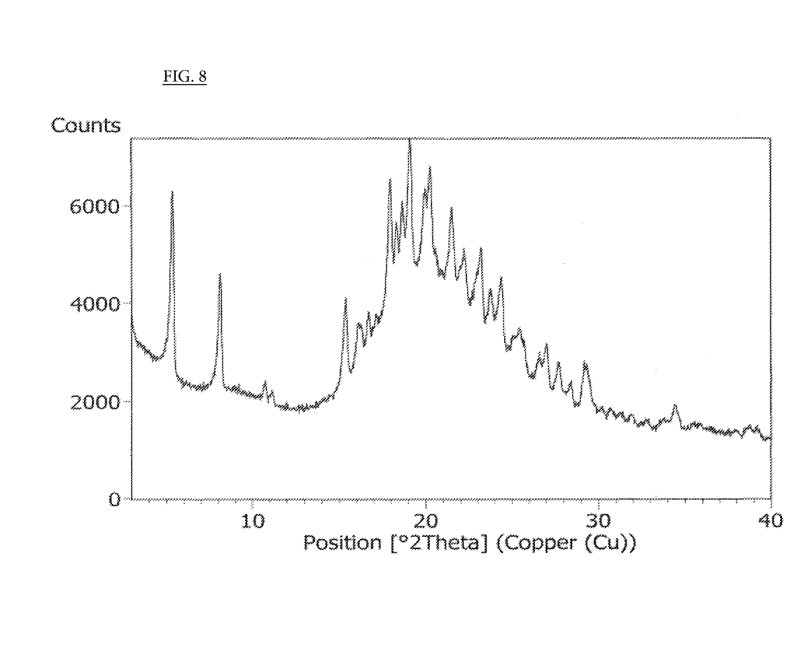

An illustrative PXRD diffractogram of the monosuccinate salt of Mirabegron is shown in FIG. 8.

The monosuccinate salt of Mirabegron may have a peak at any one or more of the values expressed in degrees 2θ given in Table 7. Although values are given in the table below, the monosuccinate salt may be defined by the claimed peaks and a particular claim may be limited to one peak only, or several peaks. The monosuccinate salt does not have to include all or even many of the peaks listed in Table 7. Some illustrative and non-limiting possible observations regarding relative intensities of the peaks are set out in Table 7.

TABLE 7
Relative peak intensities of the
monosuccinate salt of Mirabegron

| Angle (°2θ) | Relative intensity (%) |
|---|---|
| 5.35 | 100.00 |
| 8.10 | 63.65 |
| 15.34 | 41.55 |
| 17.94 | 77.50 |
| 19.09 | 82.17 |
| 20.25 | 48.21 |
| 21.51 | 38.23 |

In an embodiment, the present invention provides a p-toluenesulfonate salt of Mirabegron.

In an embodiment, the present invention provides a p-toluenesulfonate salt characterized by a PXRD diffractogram comprising peaks, in terms of degrees 2θ, at 8.1+/−0.2, 9.9+/−0.2, 17.2+/−0.2, and 18.0+/−0.2.

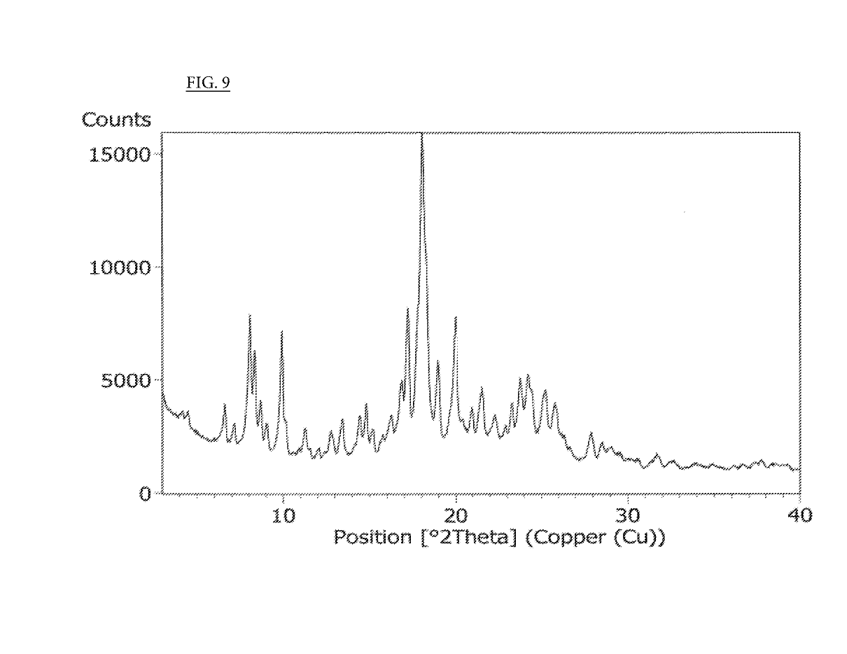

An illustrative PXRD diffractogram of the p-toluenesulfonate salt of Mirabegron is shown in FIG. 9.

The p-toluenesulfonate salt of Mirabegron may have a peak at any one or more of the values expressed in degrees 2θ given in Table 8. Although values are given in the table below, the p-toluenesulfonate salt may be defined by the claimed peaks and a particular claim may be limited to one peak only, or several peaks. The p-toluenesulfonate salt does not have to include all or even many of the peaks listed in Table 8. Some illustrative and non-limiting possible observations regarding relative intensities of the peaks are set out in Table 8.

TABLE 8
Relative peak intensities of the p-
toluenesulfonate salt of Mirabegron

| Angle (° 2θ) | Relative intensity (%) |
|---|---|
| 8.05 | 48.72 |
| 9.91 | 46.42 |
| 14.78 | 16.38 |
| 17.19 | 48.58 |
| 18.04 | 100.00 |
| 18.93 | 28.28 |
| 19.96 | 50.00 |
| 21.51 | 23.40 |

### EXAMPLES

The following examples are illustrative of some of the embodiments of the invention described herein. These examples do not limit the spirit or scope of the invention in any way.

Powder X-Ray Diffraction data were acquired on a PANanalytical X-Pert Pro MPD diffractometer with fixed divergence slits and an X'Celerator RTMS detector. The diffractometer was configured in Bragg-Brentano geometry; data was collected over a 2θ range of 3 to 40 degrees using CuKα radiation at a power of 40 mA and 45 kV. CuKβ radiation was removed using a divergent beam nickel filter. A step size of 0.017 degrees and a step time of 40 seconds was used. Samples were rotated to reduce preferred orientation effects. Samples were lightly ground prior to analysis when required.

Any form of Mirabegron should be equally suitable as a starting material for the purposes of the invention.

### Example 1

### Preparation of Form APO-1 Mirabegron

A suspension of Mirabegron (1.0 g) in (S)-(+)-1,2-propanediol (6 mL) was heated to 65° C. and maintained for about 1 hour to yield a viscous, clear, yellowish solution. The solution was removed from the heat source and methyl t-butyl ether (50 mL) was added dropwise over a period of 1 hour while the solution was allowed to cool to 20-25° C. The clear solution was then stirred for about 91 hours after which a suspension was obtained. The solid was isolated by suction filtration and dried in vacuo at 20-25° C. An off-white solid in the form of needles was obtained (700 mg). A PXRD diffractogram taken of this sample is shown in FIG. 1.

### Example 2

### Preparation of Form APO-I Mirabegron

A suspension of Mirabegron (1.0 g) in (S)-(+)-1,2-propanediol (6 mL) was heated to 65° C. and maintained for about 1 hour to yield a viscous, clear, yellowish solution. The solution was removed from the heat source and methyl t-butyl ether (35 mL) was added dropwise over a period of 1 hour while the solution was allowed to cool to 20-25° C. The solution was divided in half. One half was seeded with the product of Example 1 (1-2 mg) and the other half was used in Example 3. The half of the solution that was seeded turned turbid and was stirred for a further 3 hours after which a suspension was obtained. The solid was isolated by suction filtration and dried in vacuo at 20-25° C. for about 16 hours. An off-white solid was obtained (370 mg). The molar ratio of Mirabegron to (S)-(+)-1,2-propanediol in this sample was 1:1 by ¹H NMR. ¹H NMR (400 MHz, DMSO-d₆): δ 10.0 (s 1H), 7.49 (d, 2H), 7.30-7.27 (m, 4H), 7.23-7.20 (m, 1H), 7.12 (d, 2H), 6.89 (s, 1H), 6.3 (s, 1H), 5.30 (s, 1H), 4.7-4.5 (m, 3H), 3.44 (s, 2H), 3.5 (s, 2H), 3.3-3.1 (m, 4H), 2.8-2.6 (m, 6H), 1.7 (br s, 1H), 1.1 (d, 3H).

### Example 3

### Preparation of Form APO-1 Mirabegron

The other half of the solution obtained in Example 2 was stirred at 20-25° C. The solution was stirred for about 16 hours; it became milky after stirring for about 3 hours. The resulting suspension was isolated by suction filtration and dried in vacuo at 20-25° C. for about 16 hours. An off-white solid was obtained (300 mg).

### Example 4

### Preparation of Form APO-II Mirabegron

A suspension of Mirabegron (1.0 g) in (R)-(−)-1,2-propanediol (6 mL) was heated to 65° C. and maintained for about 30 minutes to yield a clear, yellowish solution. The solution was removed from the heat source and methyl t-butyl ether (50 mL) was added dropwise over a period of 1 hour while the solution was allowed to cool to 20-25° C. The solution was stirred for about 91 hours; it became turbid after stirring for about 40 minutes. The resulting suspension was isolated by suction filtration. A sticky solid was obtained which was washed with methyl t-butyl ether and dried in vacuo at 20-25° C. An off-white solid was obtained (110 mg). A PXRD diffractogram of this sample is shown in FIG. 2. ¹H NMR (400 MHz, DMSO-d₆): δ 10.0 (s 1H), 7.49-7.47 (d, 2H), 7.3-7.2 (m, 5H), 7.1 (d, 2H), 6.88 (s, 1H), 6.3 (s, 1H), 5.3 (brs, 1H), 4.62-4.59 (m, 3H), 3.45 (s, 2H), 3.29-3.19 (m, 4H), 2.75-2.64 (m, 6H), 1.1 (d, 3H).

### Example 5

### Preparation of Form APO-II Mirabegron

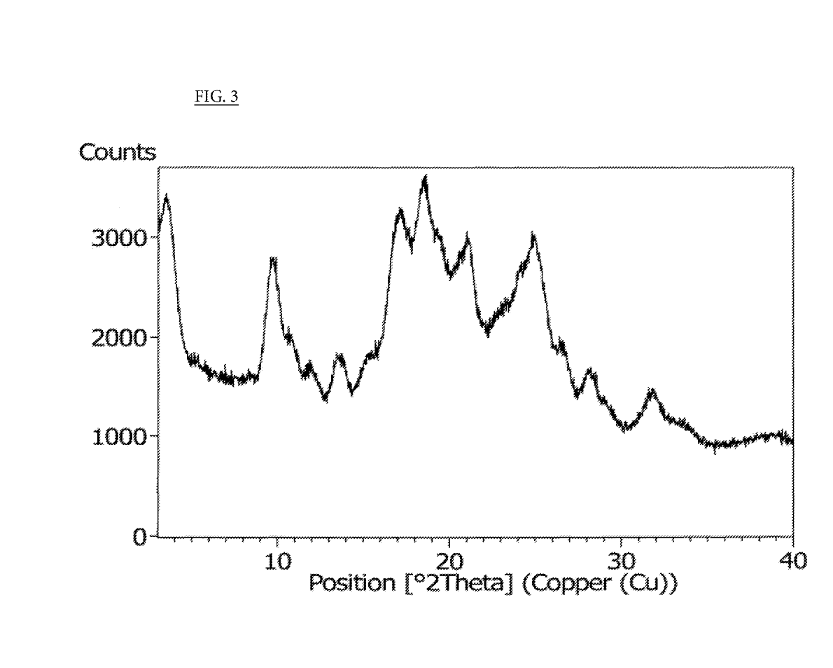

A suspension of Mirabegron (1.0 g) in (R)-(−)-1,2-propanediol (6 mL) was heated to 65° C. and maintained for about 1 hour to yield a clear, yellowish solution. The solution was removed from the heat source and methyl t-butyl ether (50 mL) was added dropwise over a period of 1 hour while the solution was allowed to cool to 20-25° C. The solution was stirred for about 16 hours. The resulting suspension was isolated by suction filtration. A sticky solid was obtained which was washed with methyl t-butyl ether (5 mL) and dried in vacuo at 20-25° C. for about 16 hours. An off-white solid was obtained (350 mg). A PXRD diffractogram of this sample is shown in FIG. 3.

### Example 6

### Preparation of Form APO-I Mirabegron

A solution of (1R)-2-{[2-(4-aminophenyl)ethyl]amino}-1-phenylethanol hydrochloride (15 g, 0.051 moles), (2-amino-1,3-thiazol-4-yl)acetic acid (8.5 g, 0.054 moles) and N-(3-dimethylaminopropyl)-N′-ethylcarbodiimide hydrochloride (11.3 g, 0.059 moles) in water was allowed to stir at 20-25° C. for 4 hours. After reaction completion, 2-methyltetrahydrofuran (150 mL) was added to the solution and the pH was adjusted to 10 using aqueous sodium hydroxide solution (11%). The two layers were separated and the organic layer was washed with water (1×45 mL). To the organic phase was added (S)-(+)-1,2-propanediol (23.4 g) in one portion. The solution was concentrated in vacuo to about 65 mL and to the clear solution was added seeds of form APO-I Mirabegron (0.15 g, 1 wt %). The solution was allowed to stir at 20-25° C. until a creamy suspension was formed (1 hour). Methyl t-butyl ether (300 mL) was charged in one portion and the suspension was allowed to stir at 20-25° C. for about 15 hours. The solid was collected by filtration and washed with methyl t-butyl ether (3×30 mL) to afford form APO-I Mirabegron (19.8 g, 82% yield).

### Example 7

### Preparation of Form APO-I Mirabegron

A solution of (1R)-2-{[2-(4-aminophenyl)ethyl]amino}-1-phenylethanol hydrochloride (10 g, 0.034 moles), (2-amino-1,3-thiazol-4-yl)acetic acid (5.9 g, 0.038 moles) and N-(3-dimethylaminopropyl)-N′-ethylcarbodiimide hydrochloride (7.5 g, 0.059 moles) in water was allowed to stir at 20-25° C. for 20 hours. After reaction completion, 2-methyltetrahydrofuran (100 mL) was added to the solution and the pH was adjusted to 9.5 using aqueous sodium hydroxide solution (11%). The two layers were separated and the organic layer was washed with water (1×30 mL). To the organic phase was added (S)-(+)-1,2-propanediol (15.6 g) in one portion. The solution was concentrated in vacuo to about 45 mL and to the clear solution was added seeds of form APO-I Mirabegron (0.10 g, 1 wt %). The solution was allowed to stir at 20-25° C. until a creamy suspension was formed (1 hour). Methyl t-butyl ether (200 mL) was charged in one portion and the suspension was allowed to stir at 20-25° C. for about 15 hours. The solid was collected by filtration and washed with methyl t-butyl ether (3×30 mL) to afford form APO-I Mirabegron (15.0 g, 81% yield).

### Example 8

### Preparation of Mirabegron Hydrobromide Salt

A solution of (1R)-2-{[2-(4-aminophenyl)ethyl]amino}-1-phenylethanol hydrochloride (5.0 g, 0.017 moles), (2-amino-1,3-thiazol-4-yl)acetic acid (2.8 g, 0.018 moles) and N-(3-dimethylaminopropyl)-N′-ethylcarbodiimide hydrochloride (3.8 g, 0.020 moles) in water was allowed to stir at 20-25° C. for 3 hours. After reaction completion, 2-methyltetrahydrofuran (50 mL) was added to the solution and the pH was adjusted to 9.5 using aqueous sodium hydroxide solution (11%). The two layers were separated and the organic layer was washed with water (1×15 mL). To the organic layer was added 48% hydrobromic acid (2.9 g, 0.017 moles) and the mixture was concentrated in vacuo to 15 mL followed by the addition of 2-methyltetrahydrofuran (50 mL). The mixture was stirred at 20-25° C. for 1 hour and the obtained suspension was filtered, washed with 2-methyltetrahydrofuran (1×10 mL) and dried to give Mirabegron hydrobromide salt (6.8 g, 83% yield). A PXRD diffractogram of this sample is shown in FIG. 4. ¹H NMR (300 MHz, DMSO-d₆): δ 10.2 (s, 1H), 9.0-8.5 (bs, 1H) 7.7-7.5 (m, 2H) 7.5-7.3 (m, 5H), 7.2-7.0 (m, 4H), 6.3 (s, 1H), 6.1 (s, 1H), 5.0 (s, 2H), 3.5 (s, 2H), 3.3-2.8 (m, 6H).

### Example 9

### Preparation of Mirabegron Monotartrate Salt

To Mirabegron free base (3.0 g, 0.01 moles) was added 2-methyltetrahydrofuran (30 mL) and DL-tartaric acid (1.1 g, 0.01 moles). The mixture was stirred at 20-25° C. for 1 hour and the obtained suspension was filtered, washed with 2-methyltetrahydrofuran (1×6 mL) and dried to afford Mirabegron monotartrate salt (3.4 g, 83% yield). A PXRD diffractogram of this sample is shown in FIG. 5. ¹H NMR (300 MHz, DMSO-d₆): δ 10.0 (s, 1H), 8.8-7.8 (bs, 1H), 7.6-7.5 (m, 2H), 7.5-7.3 (m, 5H), 7.3-7.1 (m, 2H), 6.9 (s, 2H), 6.3 (s, 1H), 4.8-4.6 (m, 1H), 4.1Hz (s, 2H), 3.4 (s, 2H), 3.2-2.8 (m, 6H).

### Example 10

### Preparation of Mirabegron Benzoate Salt

To Mirabegron free base (3.0 g, 0.01 moles) was added 2-methyltetrahydrofuran (30 mL) and benzoic acid (0.90 g, 0.01 moles). The mixture was stirred at 20-25° C. for 1 hour and the obtained suspension was filtered, washed with 2-methyltetrahydrofuran (1×6 mL) and dried to afford Mirabegron benzoate salt (3.6 g, 83% yield). A PXRD diffractogram of this sample is shown in FIG. 6. ¹H NMR (300 MHz, DMSO-d₆) 10.1 (s, 1H), 8.8-8.2 (bs, 1H), 8.1-7.8 (m, 2H), 7.7-7.2 (m, 10H), 7.2-7.0 (m, 2H), 6.9 (s, 2H), 6.3 (s, 1H), 4.9 (s, 1H), 3.4 (s, 2H), 3.2-2.7 (m, 6H).

### Example 11

### Preparation of Mirabegron Oxalate Salt

To Mirabegron free base (3.0 g, 0.01 moles) was added 2-methyltetrahydrofuran (30 mL) and oxalic acid dihydrate (0.95 g, 0.01 moles). The mixture was stirred at 20-25° C. for 1 hour and the obtained suspension was filtered, washed with 2-methyltetrahydrofuran (1×6 mL) and dried to afford Mirabegron oxalate salt (3.5 g, 88% yield). A PXRD diffractogram of this sample is shown in FIG. 7. ¹H NMR (300 MHz, DMSO-d₆) δ 10.1 (s, 1H), 9.3-8.5 (bs, 1H) 7.6-7. 5 (m, 2H), 7.5-7.3 (m, 5H), 7.2-7.1 (m, 2H), 6.9 (s, 2H), 6.3 (s, 1H), 6.1 (s, 1H), 4.9 (s, 1H), 3.4 (s, 2H), 3.3-2.8 (m, 6H).

### Example 12

### Preparation of Mirabegron Monosuccinate Salt

To Mirabegron free base (3.0 g, 0.01 moles) was added 2-methyltetrahydrofuran (30 mL) and succinic acid (0.90 g, 0.01 moles). The mixture was stirred at 20-25° C. for 1 hour and the obtained suspension was filtered, washed with 2-methyltetrahydrofuran (1×6 mL) and dried to afford Mirabegron monosuccinate salt (3.1 g, 79% yield). A PXRD diffractogram of this sample is shown in FIG. 8. ¹H NMR (300 MHz, DMSO-d₆) δ 10.0 (s, 1H), 7.6-7.4 (m, 1H), 7.4-7.2 (m, 5H), 7.2-7.1 (m, 2H), 6.9 (s, 2H), 6.3 (s, 1H), 4.8-4.6 (m, 1H), 3.4 (s, 2H), 3.0-2.7 (m, 6H), 2.3 (s, 4H).

### Example 13

### Preparation of Mirabegron p-Toluenesulfonate Salt

To Mirabegron free base (3.0 g, 0.01 moles) was added 2-methyltetrahydrofuran (30 mL) and p-toluenesulfonic acid (1.4 g, 0.01 moles). The mixture was warmed to 40° C. for 30 minutes and then stirred at 20-25° C. for 1 hour and the obtained suspension was filtered, washed with 2-methyltetrahydrofuran (1×6 mL) and dried to give Mirabegron p-toluenesulfonate salt (3.1 g, 70% yield). A PXRD diffractogram of this sample is shown in FIG. 9. ¹H NMR (300 MHz, DMSO-d₆) δ 10.1(s, 1H), 8.8-8.4 (bs, 1H) 7.7-7.5 (m, 4H), 7.5-7.3 (m, 5H), 7.3-7.0 (m, 4H), 6.9 (s, 2H), 6.3 (s, 1H), 6.1 (s, 1H), 4.9 (s, 1H), 3.4 (s, 2H), 3.3-2.8 (m, 6H), 2.3 (s, 3H).

### Example 14

### Preparation of Form APO-1 Mirabegron

The pH of a mixture of Mirabegron hydrobromide salt (6.7 g, 0.014 moles) in water (35 mL) and 2-methyltetrahydrofuran (70 mL) was adjusted to 9 with aqueous sodium hydroxide solution. The two layers were separated and the organic layer washed with water (20 mL). To the organic layer was added (S)-(+)-1,2-propanediol (20 g, 0.263 moles) and the solution concentrated to 30 mL. The solution was then seeded with form APO-1 Mirabegron (0.07 g) and stirred at 20-25° C. until a creamy suspension was formed. Methyl t-butyl ether (150 mL) was charged in one portion and the suspension was allowed to stir at 20-25° C. for about 15 hours. The solid was collected by filtration and washed with methyl t-butyl ether (3×20 mL) to afford form APO-1 Mirabegron (4.2 g, 63% yield).

### Example 15

### Preparation of Form APO-1 Mirabegron

The pH of a mixture of Mirabegron oxalate salt (3.6 g, 0.007 moles) in water (20 mL) and 2-methyltetrahydrofuran (40 mL) was adjusted to 9 with aqueous sodium hydroxide solution. The two layers were separated and the organic layer washed with water (12 mL). To the organic layer was added (S)-(+)-1,2-propanediol (11 g, 0.145 moles) and the solution concentrated to 10 mL. The solution was then seeded with form APO-1 Mirabegron (0.04 g) and stirred at 20-25° C. until a creamy suspension was formed. Methyl t-butyl ether (70 mL) was charged in one portion and the suspension was allowed to stir at 20-25° C. for about 15 hours. The solid was collected by filtration and washed with methyl t-butyl ether (3×10 mL) to afford form APO-I Mirabegron (1.8 g, 52% yield).

### Example 16

### Preparation of Form APO-I Mirabegron

The pH of a mixture of Mirabegron tartrate salt (3.8 g, 0.007 moles) in water (20 mL) and 2-methyltetrahydrofuran (40 mL) was adjusted to 9 with aqueous sodium hydroxide solution. The two layers were separated and the organic layer washed with water (12 mL). To the organic layer was added (S)-(+)-1,2-propanediol (11 g, 0.145 moles) and the solution concentrated to 10 mL. The solution was then seeded with form APO-I Mirabegron (0.04 g) and stirred at room temperature until a creamy suspension was formed. Methyl t-butyl ether (70 mL) was charged in one portion and the suspension was allowed to stir at 20-25° C. for about 15 hours. The solid was collected by filtration and washed with methyl t-butyl ether (3×10 mL) to afford form APO-I Mirabegron (2.0 g, 63% yield).

### Example 17

### Preparation of Form APO-I Mirabegron

A suspension of Mirabegron (20.0 g) in (S)-(+)-1,2-propanediol (33.8 g, 10.5 eq) and 2-methyltetrahydrofuran (50 mL) was heated to 75° C. and maintained for about 1 hour to yield a clear, yellowish solution. The solution was removed from the heat source and allowed to cool to 20-25° C. and seeded with form APO-1 (0.20 g). The resulting mixture was stirred at room temperature until a creamy suspension was obtained within 15 hours. Methyl t-butyl ether (300 mL) was added in one portion and the suspension was allowed to stir at 20-25° C. for about 3 hours. The solid was collected by filtration and washed with methyl t-butyl ether (4×40 mL) to afford form APO-I Mirabegron (18.5 g, 92% yield).

### Example 18

### Hygroscopicity Testing of Form APO-I Mirabegron

The hygroscopicity testing was conducted by placing 1 g of form APO-I Mirabegron in an unstoppered weighing vessel in a desiccator containing a saturated ammonium sulphate solution. The desiccator was placed in an oven at 25° C. for 24 hours. After this time, the percentage increase in mass was 0.89%.

In contrast, it is reported that the β-form of Mirabegron reported in CA 2 464 068 C is hygroscopic, holding moisture of up to about 3%.

### Example 19

### Solubility Testing of Form APO-I Mirabegron

Solubility testing was conducted by preparing a suspension of the subject form of Mirabegron in the indicated solvent and stirring the suspension for the indicated time period at room temperature. After this time, excess solid was removed by filtration and the volume of the supernatant was recorded. The supernatant was concentrated in vacuo and dried to constant weight to afford a solid. The solubility is shown as the weight of solid/volume of supernatant in mg/mL. The α-form and β-form of Mirabegron refer to the forms as characterized in CA 2 464 068 C. The results of this solubility testing are shown in Table 9.

TABLE 9
Solubility Data

| | | Time period | Solubility |
|---|---|---|---|
| Solvent | Form | (hours) | (mg/mL) |
| Phosphate buffer | APO-I | 2 | 6.2 |
| (pH 6.8) | α | 2 | 2.5 |
| 0.1M HCl | APO-I | 2 | 15.0 |
| | α | 2 | 13.0 |
| Distilled water | APO-I | 2 | 0.5 |
| | α | 2 | 0.2 |
| Ethanol | APO-I | 0.5 | 62.8 |
| (denatured) | α | 0.5 | 21.3 |
| | β | 0.5 | 27.2 |

Phosphate buffer was prepared by dissolving potassium dihydrogen phosphate (340 mg) and disodium hydrogen phosphate (353 mg) in deionized water (100 mL).

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) are incorporated herein by reference as if each individual priority document were specifically and individually indicated to be incorporated by reference herein and as though fully set forth herein. The invention includes all embodiments and variations substantially as hereinbefore described and with reference to the examples and drawings.

1. Form APO-I of Mirabegron wherein a Powder X-Ray Diffraction (PXRD) diffractogram comprising a peak, in terms of degrees 2θ, at 7.4+/−0.2, in addition to at least three peaks, in terms of degrees 2θ, selected from the group consisting of: 8.9+/−0.2, 16.3+/−0.2, 16.8+/−0.2, 19.8+/−0.2, 21.2+/−0.2, 24.3+/−0.2, and 25.6+/−0.2.

2. (canceled)

3. The form APO-I of Mirabegron of claim 1, wherein the PXRD diffractogram further comprises peaks, in terms of degrees 2θ, 8.9+/−0.2, 16.3+/−0.2, 16.8+/−0.2, and 24.3+/−0.2.

4. The form APO-I of Mirabegron of claim 1, wherein the PXRD diffractogram is substantially similar to the diffractogram as depicted in FIG. 1.

5. The form APO-I of Mirabegron of claim 1, wherein the form APO-I is a solvate with (S)-(+)-1,2-propanediol having a molar ratio of Mirabegron to (5)-(+)-1,2-propanediol of 1:1.

6. Form APO-II of Mirabegron wherein a Powder X-Ray Diffraction (PXRD) diffractogram comprising peaks, in terms of degrees 2θ, at 9.7+/−0.2, 15.5+/−0.2, 17.1+/−0.2, and 18.5+/−0.2 and at least one peak selected from the group consisting of: 12.3+/−0.2 and 13.8+/−0.2.

7. The form APO-II of Mirabegron of claim 6, wherein the PXRD diffractogram is substantially similar to the diffractogram as depicted in FIG. 2.

8. The form APO-II of Mirabegron of claim 6, wherein the form APO-II is a solvate with (R)-(−)-1,2-propanediol having a molar ratio of Mirabegron to (R)-(−)-1,2-propanediol of 1:1.

9. A process for the preparation of form APO-I of Mirabegron comprising treating a mixture of Mirabegron in a solvent comprising (S)-(+)-1,2-propanediol with an antisolvent to provide form APO-I of Mirabegron.

10. (canceled)

11. The process of claim 9, wherein the mixture of Mirabegron is in the form of a suspension of Mirabegron in a solvent comprising (S)-(+)-1,2-propanediol that is seeded with form APO-I of Mirabegron.

12. The process of claim 9, wherein the solvent is a mixture of (S)-(+)-1,2-propanediol and one or more solvents selected from the group consisting of ketones, esters, cyclic ethers, and n-butanol.

13. The process of claim 12, wherein the solvent is a mixture of (S)-(+)-1,2-propanediol and one or more solvents selected from the group consisting of methyl isobutyl ketone, ethyl acetate, isopropyl acetate, 2-methyltetrahydrofuran and n-butanol.

14. (canceled)

15. The process of claim 9, wherein the antisolvent is methyl t-butyl ether.

16. The process of claim 11, wherein the solvent is a mixture of (S)-(+)-1,2-propanediol and 2-methyltetrahydrofuran, the antisolvent is methyl t-butyl ether, and the treating is done at a temperature between 20° C. and about 30° C.

17.-30. (canceled)
